(19) United States
(12) Patent Application Publication
(10) Pub. No.: US 2007/0082071 A1
(43) Pub. Date: Apr. 12, 2007

(54) Composition for controlling the respiratory effects of inhaled pollutants & allergens

(76) Inventor: John A. Willimann, Coral Gables, FL (US)

Correspondence Address: ROBERT M. DOWNEY, P.A., 6751 N. FEDERAL HWY., SUITE 300, BOCA RATON, FL, 33487, US

(21) Appl. No.: 11/545,720

(22) Filed: Oct. 10, 2006

Related U.S. Application Data
(60) Provisional application No. 60/725,050, filed on Oct. 7, 2005.

(51) Int. Cl. A61K 36/889; A61K 36/752; A61K 36/53; A61K 36/534; A61K 36/48; A61K 36/42; A61K 36/28

(52) U.S. Cl. 424/727; 424/745; 424/757; 424/747; 424/736; 424/758; 424/764

(57) ABSTRACT

An anti-inflammatory composition for application to the nasal mucosa includes a combination of essential oils. The composition is intended to help prevent and/or alleviate the effects of exposure caused by the inhalation of pollutants and allergens. Preferred embodiments of the composition include jojoba oil, rosemary oil, any of a variety of citrus oils, coconut oil, sesame oil, soy oil, thyme oil, oregano oil, chamomile oil, peppermint oil, cardiospermum halicacabum, galphimia glauca, luffa operculata, bee's milk, bee's wax, and aloe vera in various combinations and sub-combinations. The composition may further include lauric acid, d-limonene and luteolin.

### BACKGROUND OF THE INVENTION

This application is based on provisional patent application Ser. No. 60/725,050 filed on Oct. 7, 2005.

1. Field of the Invention

This invention relates to an anti-inflammatory composition for application to the nasal mucosa to control the inflammatory response in the nasal tissues often caused by inhalation of various pollutants and allergens and includes a combination of essential oils.

2. Discussion of the Related Art

Allergies and chronic rhinitis are generally characterized by the excess proliferation of immune responder cells in the bloodstream and nasal fluids. The influx of responder cells to the airways and nasal passages produces the congestion, fluid outpouring and swelling that many allergy sufferers find so debilitating. While many antihistamines lessen some of these symptoms by preventing the release of histamine from mast cells or blocking histamine receptor sites, they do nothing to prevent the excess activation or proliferation of immune responder cells. The effects of pollution (ground level ozone, smog, sulfur dioxide, nitrogen oxide, and suspended particulates) in the air is believed to be responsible for a multitude of respiratory, as well as, cardiovascular ailments.

Allergic rhinitis, commonly referred to as “hay fever”, is believed to be a response to allergens such as pollens and molds and might be exacerbated by the exposure to pollution (ozone, NO, SO₂, particulates). Primary sources for outdoor allergens include vascular plants (pollen, fern spores, soy dust), and fungi (spores, hyphae) with nonvascular plants, algae, and arthropods contributing smaller numbers of allergen-bearing particles.

Respiratory allergic diseases include seasonal allergic rhinitis or hay fever, perennial allergic rhinitis and allergic asthma. While the prevalence of allergies and associated conditions is difficult to accurately assess, recent estimates show that between 20% to 25% of the U.S population is afflicted with allergic rhinitis. It is believed that allergic rhinitis is currently the most common of all chronic diseases in children. Unfortunately, untreated allergic rhinitis not only detrimentally affects children's physical and psychosocial well-being, quality of life, and capacity to function and learn, but it is also associated with, and may contribute to, potentially serious conditions, including asthma and sinusitis.

In recent years, scientists have shown that air pollution from cars, factories and power plants is a major cause of chronic rhinitis, asthma and allergy attacks. More than 159 million Americans—over half the nation's population—live in areas with polluted air. A research study published in 2002 estimated that 30 percent of childhood asthma and allergic episodes are due to environmental exposures, costing the US more than $2 billion per year. Studies also suggest that air pollution may contribute to the development of asthma and allergic rhinitis in previously healthy people.

The following sources of air pollutants can trigger asthma and allergic rhinitis:
Ground Level Ozone: A toxic component of smog, ozone triggers asthma attacks and makes existing asthma worse. It may also lead to the development of asthma in children. Ozone is produced at ground level when tailpipe pollution from cars and trucks reacts with oxygen and sunlight. Ground level ozone is a big problem in cities with lots of traffic, such as Los Angeles, Houston and New York City. In 2004, according to the American Lung Association, 136 million people lived in areas that violated ozone air quality standards.
Sulfur Dioxide (SO₂): A respiratory irritant associated with the onset of asthma and allergy attacks, sulfur dioxide is produced when coal and crude oil are burned. Coal-fired power plants, particularly older plants that burn coal without SO₂ pollution controls, are the worst SO₂ polluters. One in five Americans lives within 10 miles of a coal-fired power plant. Oil refineries and diesel engines that burn high-sulfur fuel also release large amounts of SO₂ into the air.
Particulate Matter: This term refers to a wide range of pollutant such as dust, soot, fly ash, diesel exhaust particles, wood smoke and sulfate aerosols which are suspended as tiny particles in the air. Some of these fine particles can become lodged in the lungs and often trigger allergy and asthma attacks. Studies have shown that the number of hospitalizations for asthma increases when levels of particulate matter in the air rise. Coal-fired power plants, factories and diesel vehicles are major sources of particulate pollution. Around 81 million people live in areas that fail to meet national air quality standards for particulate matter in the United States alone.

Nitrogen Oxide (NOx): A gas emitted from tailpipes and power plants, nitrogen oxide contributes to the formation of ground-level ozone and smog. It also reacts with other air pollutants to form small particles that can cause breathing difficulties, especially in people with asthma and allergic rhinitis.

### SUMMARY OF THE INVENTION

The present invention is directed to a composition comprising a combination of several essential oils for controlling inhalation of pollutants and allergens. Preferred embodiments of the composition include Jojoba oil, rosemary oil, any of a variety of citrus oils, sesame oil, soy oil, thyme oil, oregano oil, chamomile oil, peppermint oil, cardiospermum halicacabum, galphima glauca, luffa operculata, bee's milk, bee's wax, and aloe vera in various combinations and sub-combinations. The composition may further include lauric acid, d-limonene and luteolin. When the composition is applied to the nasal mucosa, these oils provide a physical barrier that acts as a target for oxidizing and chemically-active pollution components, reducing these effects on the epithelial cells that line the nasal mucosal surfaces. Anti-inflammatory benefits may also be provided by the composition through its capacity to up-regulate anti-oxidant and anti-inflammatory gene pathways, including, but not limited to, those of Nrf2, Heme Oxygenase-1, Catalase, the Glutathione redox system, Superoxide Dismutase, Quinone Reductase, Thioredoxin, and Toll-like Receptor-4. The composition may also provide protection by down-regulating gene pathways and their enzyme products that are pro-inflammatory and pro-oxidant. Examples include, but are not limited to, Nuclear Factor kappa B, Activator Protein-1, and inducible Nitric Oxide Synthase pathways. In addition, Rosmarinic acid, a plant polyphenol derived from rosemary leaf and present in rosemary oil, has been shown to provide effective, temporary relief of allergy symptoms. Rosmarinic acid, along with the other oils mentioned above, have significant antioxidant, anti-inflammatory and even some antimicrobial activities. The antioxidant activity of rosmarinic acid is known to be stronger than that of vitamin E. Rosmarinic acid, as well as other of the aforementioned oils, helps to prevent cell damage caused by free radicals and also provides anti-inflammatory properties. And, unlike antihistamines, these oils help to prevent the activation of immune responder cells, which cause swelling and fluid formation. Scientists have demonstrated that perilla leaf enriched with rosmarinic acid provided significant relief from seasonal allergies by inhibiting polymorphonuclear leukocyte infiltration into the nostrils. Unlike antihistamines, the composition of the present invention prevents the activation of immune responder cells by trapping many allergens and pollutants that seek to enter the body through the nose. The composition also protects the cells from the harmful effects of oxidizing agents such as those mentioned supra (i.e., ozone, nitrogen oxide, sulfur dioxide, and particulate matter.)

The composition of the present invention helps to alleviate the body's immunologic response to many allergens including, but not limited to, dust mites, pollen, hay fever, animal dander, dust, particulate pollution, ozone, sulfur dioxide, and nitrogen oxide. In particular, the composition is effective in trapping these allergens and alleviating the body's response to their presence. In the case of ozone, SO₂ and NOx, the composition acts as a barrier in the nose and lessens the IGA response in the body. It is also believed that the composition works to lessen the Interleukin expression in the nose, especially IL8, which is commonly responsible for the inflammatory response in the nasal cavity. A particularly effective formulation includes one or more of the following ingredients: jojoba oil, peppermint oil, rosemary oil, citrus oils, sesame oil, soy oil, thyme oil, coconut oil, oregano oil and chamomile oil. The composition may further include lauric acid, d-limonene, and aloe vera. In addition, the composition may include luteolin, another plant flavonoid that has been shown to maintain normal respiratory function and healthy fluid balance.

Objects and Advantages

Considering the foregoing, it is a primary object of the present invention to provide a composition for application to the nasal mucosa for controlling the respiratory effects of inhaled pollutants and allergens.

It is a further object of the present invention to provide a composition comprising a combination of essential oils for application to the nasal mucosa, wherein the composition is effective in trapping allergens and alleviating the body's response to their presence.

It is a further object of the present invention to provide a composition comprising a combination of essential oils for controlling inhalation of pollutants and allergens, and wherein the composition possesses anti-inflammatory and anti-oxidant properties.

It is a further object of the present invention to provide a composition comprising a combination of essential oils, wherein the composition interferes with the release or effectiveness of agents produced by bacterial, fungal or other biological contaminants that enter the nasal passages along with allergens and particulate matter.

It is a further object of the present invention to provide a composition comprising a combination of essential oils, wherein the composition masks or reduces the irritating or objectionable odors associated with exposure to airborne pollutants in indoor and outdoor environments.

It is a further object of the present invention to provide a composition comprising a combination of essential oils, wherein the composition interferes with the release and downstream pathway effects of chemicals released by cells involved with the immune/allergic response.

### DETAILED DESCRIPTION OF THE PREFERRED EMBODIMENTS

The present invention is directed to a long lasting anti-inflamatory composition for application to the nasal mucosa. The anti-inflammatory composition of the present invention incorporates the use of one or more essential oils. Other oils and agents are contemplated for use in the composition as the anti-inflammatory solution, either alone or as a combination.

One or more primary ingredients of the composition are present according to the following percentages by weight of the composition:

| | Amount |
|---|---|
| Ingredients | (% by Weight of the Composition) |
| Peppermint oil | between 0.1% and 5% |
| Jojoba oil | between 5% and 70% |
| Soy oil | between 0.5% and 56% |
| Cocos Nucifera (coconut oil) | between 1% and 30% |
| Citrus oil | between 0.5% and 15% |
| Rosemary oil | between 0.5% and 20% |

The anti-inflammatory composition of the present invention may include the following additional ingredients, alone or in combination: lauric acid; d-limonene; sesame oil; chamomile oil; cardiospermum halicacabum; galphimia glauca, luffa operculata; bees milk; and a preservative such as benzylkonium chloride or BHT or sodium benzoate. Other additional ingredients of the composition may be present according to the following percentages by weight of the composition:

| | Amount |
|---|---|
| Additional Ingredients | (% by Weight of the Composition) |
| Bee's Milk | between .2% and 50% |
| Bee's Wax | between .5% and 10% |
| Cardiospermum halicacabum | between .2% and 5% |
| Galphimia glauca | between .2% and 5% |
| Luffa operculata | between .2% and 5% |
| Chamomile oil | between 1% and 10% |

A further embodiment of the composition has been proven to help alleviate the body's immunologic response to many allergens and pollutants. The following ingredients have been found to be effective in the composition when present according to the following percentages by weight of the composition:

| | Amount |
|---|---|
| Ingredients | (% by Weight of the Composition) |
| Bee's Wax | between 0.01% and 30% |
| Bee's Milk | between 0.01% and 30% |
| Fruit Wax | between 0.1% and 5% |

The following examples demonstrate various combinations of ingredients, including the essential ingredients, which have been observed to yield:

In one series of experiments using the test combination of ingredients according to the several examples of the composition listed below, epithelial cells of the human respiratory tract were utilized to test the protective effects of the preparation against oxidant damage caused by exposure to the environmental air pollutant, ozone. The cells were grown in culture at the air-liquid interface to allow them to undergo mucociliary differentiation to the cellular anatomy normally seen in vivo. Cells thus cultured grow on porous filters with medium below and their apical surfaces exposed to the air above. In one set of experiments, the apical surfaces of the cells were treated for 5 minutes with the test composition or with culture medium (control), which was immediately removed. The cultures were then transferred to environmentally controlled chambers for exposure to air, or air containing 0.20 ppm ozone, for 3 hours. The release of Tumor Necrosis Factor alpha (TNFα) was used as a marker of oxidant stress in the cells following ozone exposure. TNFα release was reduced by 89.7% post exposure in test composition-treated cells compared to those treated with culture medium. These results support the claim that application of the test composition provides immediate protection against the toxic effects of oxidant pollutants, such as ozone. In another set of experiments, cells were similarly pretreated with the test composition or medium control, but were held for three hours prior to exposure to ozone following the above protocol. In these experiments, TNFαrelease was completely abolished in test composition-treated cells compared to those treated with culture medium. These results support the claim that the test composition activates time-dependent changes within the cells, likely through anti-oxidant and anti-inflammatory pathways, that afford protection from exposure to agents that activate or cause damage to cells through oxidant pathways.

Examples of the test composition are as follows:

### EXAMPLE 1

| | Amount |
|---|---|
| Ingredient | (% by Weight of the Composition) |
| Peppermint oil | .5% |
| Aloe Vera oil | 4.5% |
| Soy oil | 70% |
| Cocos Nucifera | 20% |
| Citrus Oil | 4.9% |
| Benzalkonium chloride | .1% |

### EXAMPLE 2

| | Amount |
|---|---|
| Ingredient | (% by Weight of the Composition) |
| Peppermint oil | .5% |
| Cocos Nucifera | 20% |
| Citrus Sinensis | 9.4% |
| Glycine Soja | 70% |
| Benzylkonium Chloride | 0.1% |
| (preservative) | |

### EXAMPLE 3

| | Amount |
|---|---|
| Ingredient | (% by Weight of the Composition) |
| Jojoba oil | 34.8% |
| Cocos Nucifera | 3% |
| Citrus Sinensis | 2% |
| Aloe Vera | 2.85% |
| Peppermint oil | .25% |
| Glycine Soja | 57% |
| BHT | 0.1% |

### EXAMPLE 4

| | Amount |
|---|---|
| Ingredient | (% by Weight of the Composition) |
| Glycine Soja | 35% |
| Citrus Sinensis | 30% |
| Cocos Nucifera | 30% |
| Bees Milk | 4.9% |
| Preservative | 0.1% |

### EXAMPLE 5

| | Amount |
|---|---|
| Ingredient | (% by Weight of the Composition) |
| Bees Milk | 50% |
| Lecithin | 10% |
| Citrus Sinensis | 20% |
| Glycine Soja | 19.9% |
| Preservative | 0.1% |

In one of its applications, the anti-inflammatory and anti-oxidant composition is typically administered to the nasal mucosa with the use of a metered nasal spray applicator according to the following procedure:

1. The bottle containing the composition is shaken well to ensure complete mixing of the ingredients.

2. The tip of the applicator nozzle is inserted into one nostril.

3. While breathing in gently through the nose, the applicator is activated to release a fine spray, in a volume from 10 to 200 microliters, into the nasal cavity.

4. Step 3 is repeated for the second nostril.

In another of its applications, the composition is applied to the vestibule of each nostril using a cotton swab applicator according to the following procedure:

1). Shake the bottle (containing the composition) well to insure complete mixture of the ingredients.

2) Apply approximately 4 drops of the composition to the cotton tip of a cotton swab so that the cotton tip is fully saturated with the composition.

3). Place the thumb and index finger on the swab stem directly below the wetted cotton tip of the swab. Prepare to apply the composition to the rim of each nostril just past the nasal opening.

4). Place only the cotton tip of the swab just inside of the nostril opening. Using a gentle motion, make 3 or 4 circles to fully apply the composition to the rim of the nostril. Repeat this step for the other nostril.

5). Discard the swab. Gently squeeze the nostrils together to ensure even distribution of the solution about the rim surrounding each nostril opening.

In order to evaluate the anti-inflammatory efficacy of one sample of the composition when applied to the nasal mucosa of human volunteers by spray application, a study was conducted at the National Institute of Health in Manila, Philippines. Modifications to standard methods for several procedures required for the study, such as nasal lavage, cytologic preparation of lavaged cells for differential analysis and the isolation of lavage fluid for quantification of markers of inflammation by enzyme-linked immunosorbent assay (ELISA), were validated in laboratories at the Johns Hopkins Bloomberg School of Public Health in Baltimore, Md. The details of the Manila study are set forth below.

Purpose:

The study was designed to evaluate the effectiveness of the test composition, when applied by spray aerosol, in reducing measures of the inflammatory and irritating effects of ambient air pollution on the upper respiratory system. Study endpoints included both quantitative and qualitative measures of this effectiveness.

Scope and Design:

Following a double-blinded, randomized cross-over design, the study recruited and enrolled 45 subjects who worked as traffic enforcers for the Metropolitan Manila Development Authority. This group of subjects represents a cohort of individuals who are very highly exposed to primarily vehicular diesel exhaust and related street-level airborne gaseous and particulate pollutants on a daily basis. After screening to exclude smokers and those with acute or chronic respiratory diseases or specific allergies, informed consent was obtained and the subjects were randomly assigned to two groups, to start in either the test composition arm or the placebo arm. Both preparations were applied by metered spray in a volume of 50 microliters to each nostril. The study encompassed a three week period, comprising one week of three-times daily application of the test composition or placebo (Arm 1), one week of wash-out, and a final week of three-times daily application of the second of the two preparations (Arm 2). Subjects completed daily symptom questionnaires during each of the treatment arms and, at the beginning and end of each arm, subjects were interviewed and underwent nasal lavage to allow measurement of the levels of cellular inflammation and the release of inflammatory mediators. Interviews and internal controls were used to verify compliance with the treatment protocol. No reportable Adverse Events were observed during or following completion of the study.

While the composition of the present invention has been described and exemplified according to several preferred embodiments thereof, it is recognized that departures from the instant disclosure are fully contemplated within the spirit and scope of the invention which is not to be limited except as defined in the following claims as interpreted under the Doctrine of Equivalents.

What is claimed is:

1. An anti-inflamatory composition consisting of:
rosemary oil;
peppermint oil;
jojoba oil;
coconut oil;
soy oil; and
citrus sinensis.

2. The composition as recited in claim 1 further including at least one oil selected from the group consisting of:
sesame oil;
chamomile oil;
oregano oil; and
thyme oil.

3. The composition as recited in claim 1 further including at least one ingredient selected from the group consisting of:
cardiospermum halicacabum;
galphimia glauca; and
luffa operculata.

4. The composition as recited in claim 1 further including at least one ingredient selected from the group consisting of:
lauric acid;
d-limonene; and
luteolin.

5. The composition as recited in claim 1 further including at least one ingredient selected from the group consisting of:
bee's wax;
bee's milk; and
fruit wax.

6. An anti-inflamatory composition for application to the nasal mucosa consisting of:
rosemary oil in an amount of between 0.5% and 20.0% by weight of the composition;
peppermint oil in an amount of between 0.1% and 5.0% by weight of the composition;
jojoba oil in an amount of between 5.0% and 70.0% by weight of the composition;
coconut oil in an amount of between 1.0% and 30.0% by weight of the composition;
soy oil in an amount of between 0.5% and 56.0% by weight of the composition; and
citrus sinensis in an amount of between 0.5% and 15% by weight of the composition.

7. The composition as recited in claim 6 further including at least one ingredient selected from the group consisting of:
bee's wax;
bee's milk; and
fruit wax.

8. The composition as recited in claim 6 further comprising:
bee's wax in an amount of between 0.01% and 30.0% by weight of the composition;
bee's milk in an amount of between 0.01% and 30.0% by weight of the composition; and
fruit wax in an amount of between 0.1% and 5.0% by weight of the composition.

9. The composition as recited in claim 6 further comprising:
a preservative.

10. The composition as recited in claim 6 further comprising:
aloe vera oil in an amount of between 2.85% and 4.5% by weight of the composition.

11. An anti-inflamatory composition for application to the nasal mucosa including at least two oils selected from the group consisting of:
rosemary oil;
peppermint oil;
jojoba oil;
coconut oil;
soy oil; and
citrus sinensis.
